(19) United States
(12) Patent Application Publication
(10) Pub. No.: US 2012/0184628 A1
(43) Pub. Date: Jul. 19, 2012

(54) O/W TYPE COSMETIC COMPOSITION WITH IMPROVED DOSAGE FORM STABILITY

(76) Inventors: Jung Yeob Jeong, Yongin-si (KR); Young So Kim, Yongin-si (KR); Sung Il Park, Seoul (KR); Youn Joon Kim, Seoul (KR); Sang Hun Han, Suwon-si (KR)

(21) Appl. No.: 13/499,448

(22) PCT Filed: Sep. 17, 2010
(86) PCT No.: PCT/KR2010/006379

§ 371 Date: Mar. 30, 2012

(30) Foreign Application Priority Data
Sep. 30, 2009 (KR) 10-2009-0093273

(51) Int. Cl. A61K 8/81

(52) U.S. Cl. 514/772.6

(57) ABSTRACT

The present invention relates to an O/W type cosmetic composition with improved formulation stability, and more particularly, to an O/W type cosmetic composition comprising a branched polymer having a lipophilic alkyl side chain and an anionic surfactant, as active ingredients for improving formulation stability.

### TECHNICAL FIELD

The present invention relates to an O/W type cosmetic composition with improved formulation stability, and more particularly, to an O/W type cosmetic composition comprising a branched polymer having a lipophilic alkyl side chain and an anionic surfactant as active ingredients for improving formulation stability.

### BACKGROUND ART

Cosmetic creams are classified based on their formulation and physical state. Especially, the cosmetic cream with a cream hardness of 40 dyne/cm² (T.Speed 2 cm/min) or above is normally defined as high-hardness cream.

Generally, the cosmetics with high-hardness cream formulation contain a combination of solid ingredients, such as aliphatic alcohol or wax, and water-soluble polymers, which are the main components determining the viscosity of the cream. The cream formulation looks rich and nutritious and gives a heavy and sticky feeling during use due to a large content of aliphatic alcohol and wax, which are susceptible to temperature change, requiring a cautious handling in manufacture and deteriorating formulation and temporal stabilities. As for cosmetics containing silicon oil, which are incompatible with oil and water phases, the formulation stability deteriorates with an increase in the content of the silicon oil. The composition using water-soluble polymers alone gives a lubricious texture with poor absorptivity to the skin. In particular, polymers are an essential ingredient necessary to modern cosmetics that controls the viscosity of the contents to enhance the user's aesthetic sense and secures the emulsion stability of oil particles and the dispersion stability of dispersed materials. Using an excess of polymers is needed to acquire desired functions sufficiently, in which case the formulation applied on the skin leaves the skin lubricious and, upon evaporation of water, produces polymers coming off the skin like dirt or dead skin so that it cannot be spread uniformly on the skin in the subsequent makeup step. To overcome this problem, different types of water-soluble polymers have been developed, only with little improving effects.

The cosmetic cream composition, which normally contains a hydrophilic nonionic surfactant and a higher aliphatic alcohol, is excellent in temporal stability and workability but possibly becomes deteriorated in formulation stability according to on the ratio of the surfactant to the higher aliphatic alcohol, thereby requiring a cautious handling in manufacture and causing adverse effects on the skin.

### DISCLOSURE OF INVENTION

### Technical Problem

Accordingly, in an attempt to disclose the interaction between a specific polymer and an anionic surfactant and to prepare a high-hardness cosmetic composition with good formulation stability using the interaction, the inventors of the present invention have found it out that using a combination of a branched polymer having a lipophilic alkyl side chain, such as an acrylate/C10-30 alkyl acrylate crosspolymer, and an anionic surfactant, such as potassium cetyl phosphate, at a predetermined ratio enhances the hardness of the cosmetic composition according to the interaction between the polymer and the anionic surfactant to maintain the highest stability.

It is therefore an object of the present invention to provide an O/W type cosmetic composition having enhanced hardness and maintaining the high stability.

### Technical Solution

To accomplish the above object, according to the present invention, there is provided an O/W type cosmetic composition containing a branched polymer having a lipophilic alkyl side chain and an anionic surfactant as active ingredients for improving formulation stability.

### Advantageous Effects

The O/W type cosmetic composition of the present invention uses a combination of a polymer having a lipophilic alkyl side chain and an anionic surfactant to enhance the hardness, thereby improving formulation stability.

### BEST MODE FOR CARRYING OUT THE INVENTION

The present invention is directed to an O/W type cosmetic composition comprising, as a formulation stabilizer, a combination of a branched polymer having a lipophilic alkyl side chain and an anionic surfactanct. In one example of the present invention, the O/W type cosmetic composition may be a high-hardness cosmetic cream.

In the present invention, the branched polymer having a lipophilic alkyl side chain may be an acrylate/C10-30 alkyl acrylate crosspolymer, an acrylate/C12-22 alkyl methacrylate copolymer, an acrylate/beheneth-25 methacrylate copolymer, or an acrylate/ceteth-20 methacrylate copolymer. Among these polymers, an acrylate/C10-30 alkyl acrylate crosspolymer is preferred as the branched polymer of the present invention.

The anionic surfactant as used herein may be potassium cetyl phosphate, aluminum isostearyl glyceryl phosphate, or sodium lauryl sulfate, which anionic surfactants contain: an anionic group as a hydrophilic group, such as of phosphate, sulphate, or citrate; a counter ion, such as sodium ion, potassium ion, or ammonium ion; and a hydrophobic group such as a C8-C24 aliphatic alkyl group. The most preferred anionic surfactant is potassium cetyl phosphate.

The O/W type cosmetic composition of the present invention may contain, with respect to the total weight of the composition, 0.05 to 0.5 wt. % of the branched polymer, and 0.5 to 2 wt. % of the anionic surfactant. The content of the branched polymer less than 0.05 wt. % results in a failure to achieve a thickening effect, while the content of the branched polymer greater than 0.5 wt. % causes a safety problem to the skin. The content of the anionic surfactant less than 0.5 wt. % causes a failure in emulsification of particles, while the content of the anionic surfactant greater than 2 wt. % leads to a safety problem such as skin irritation.

The O/W type cosmetic composition of the present invention features a high level of cream hardness 40 dyne/cm² (T.Speed 2 cm/min) or above. Therefore, the novel O/W type cosmetic composition substantially changes cream hardness and sensory attributes to enhance formulation stability. Moreover, the O/W type cosmetic composition does not deteriorate in regard to formulation stability even when using a greater quantity of oil or wax generally added in cosmetic creams.

The O/W type cosmetic composition of the present invention may contain, with respect to the total weight of the composition, 2 to 30 wt. % of an oil, which oil is not specifically limited to and may include silicon oil, such as dimethicone, cyclomethicone, etc.; ester-based oil, such as cetyl ethyl hexanoate, C12-15 alkyl benzoate, etc.; hydrocarbon-based oil, such as hydrogenated polydecene, squalane, etc.; or a mixture of these. The term “oil content” as used herein refers to the total weight of oily ingredients not containing an emulsion stabilizer.

The O/W type cosmetic composition of the present invention may also contain, with respect to the total weight of the composition, 0 to 20 wt. % of auxiliary components generally used in manufacture of cosmetics, such as pigment, fragrance, preservative, thickener, and so forth.

The O/W type cosmetic composition of the present invention is applicable to a cream formulation, which is safest and most reasonable, or other formulations by way of adjusting the content of each ingredient, such as, for example: basic skin care cosmetics (e.g., skin softener, essence lotion, face lotion, cream, facial mask, gel, patch, etc.; color cosmetics (e.g., lipstick, makeup base, makeup powder, etc.; cleansers (e.g., shampoo, conditioner, body cleanser, toothpaste, mouthwash, etc.; hair fixatives (e.g., hair toner, hair gel, etc.); hair care products (e.g., hair restorer, hair dye, etc.); or a wider range of applications including drugs and quasi-drugs (e.g., lotion, ointment, gel, cream, patch, or spray).

Hereinafter, the present invention will be described in further detail with reference to the following examples and experimental examples, which are not only for illustrative purposes and are not intended to limit the scope of the present invention. It is also to be understood that variations, replacements, and insertions may be resorted to as will be apparent to those skilled in the art within the scope of the present invention.

### Example 1 and Comparative Examples 1 to 5

O/W type cosmetic creams of Example 1 and Comparative Examples 1 to 5 were prepared according to the compositions of Table 1 and the following preparation method.

TABLE 1

| | | Example | Comparative Example | | | | |
|---|---|---|---|---|---|---|---|
| Div. | Ingredient | 1 | 1 | 2 | 3 | 4 | 5 |
| Oil phase | Glycerin stearate | 1 | 1 | 1 | 1 | 1 | 1 |
| | Behenyl alcohol | 3.5 | 3.5 | 3.5 | 3.5 | 3.5 | 3.5 |
| | Cetearyl alcohol | 1.5 | 1.5 | 1.5 | 1.5 | 1.5 | 1.5 |
| | Dicarprylyl carbonate | 5 | 5 | 5 | 5 | 5 | 5 |
| | Shear butter | 3 | 3 | 3 | 3 | 3 | 3 |
| | Cetyl ethyl hexanoate | 3 | 3 | 3 | 3 | 3 | 3 |
| | Dimethicone | 5 | 5 | 5 | 5 | 5 | 5 |
| | Potassium cetyl | 1 | 0 | 0 | 1 | 1 | 1 |
| | phosphate | | | | | | |
| | Polyglyceryl-3 | 0 | 1 | 0 | 0 | 0 | 0 |
| | methylglucose | | | | | | |
| | distearate | | | | | | |
| | PEG-40 stearate | 0 | 0 | 1 | 0 | 0 | 0 |
| Water | Purified water | Balance | | | | | |
| phase | Disodium EDTA | 0.02 | 0.02 | 0.02 | 0.02 | 0.02 | 0.02 |
| | Butylene glycol | 5 | 5 | 5 | 5 | 5 | 5 |
| | Phenoxy ethanol | 0.3 | 0.3 | 0.3 | 0.3 | 0,3 | 0.3 |
| Thickener | Acrylate/C10-30 alkyl | 0.25 | 0.25 | 0.25 | 0 | 0 | 0 |
| | acrylate crosspolymer | | | | | | |
| | Carbomer | 0 | 0 | 0 | 0.25 | 0 | 0 |
| | Xanthan gum | 0 | 0 | 0 | 0 | 0.25 | 0 |
| | Polyacrylate-13 & | 0 | 0 | 0 | 0 | 0 | 0.25 |
| | polyisobutene & | | | | | | |
| | polysorbate 20 | | | | | | |

### Preparation Method

1) The oil phase was heated to 70° C.

2) The water phase was blended with an agi-mixer and heated at 75° C. until melted.

3) The oil phase was added to the water phase, and the mixture was agitated with a homogenizer at 8,000 rpm for 5 minutes to form a cream.

4) An additive such as a thickener was added to the cream, which was then agitated with the homogenizer at 8,000 rpm for 3 minutes.

5) After completion of agitation, the cream was removed of bubbles and then cooled down to 30° C.

### Experimental Example 1

### Applications Based on Change of Polymer and Surfactant

The O/W type cosmetic creams of Example 1 and Comparative Examples 1 to 5 were measured in regard to the change of cream hardness depending on the types of the polymer and the surfactant. The hardness variations were determined for the test materials (compositions of Comparative Examples 1 to 5) with reference to the composition of Example 1. The results are presented in Table 2. The cream hardness was measured at a full scale pressure of 2 kg and a scale of 2 cm/2 min with COMPAC-100 2^{ND} supplied by SUN RHEOMETER.

TABLE 2
Change of Cream Hardness

| | Hardness | Hardness variation |
|---|---|---|
| Test material | (dyne/cm²) | (dyne/cm²) |
| Example 1 | 55 | 0 |
| Comparative Example 1 | 30 | −20 |
| Comparative Example 2 | 25 | −30 |
| Comparative Example 3 | 40 | −15 |
| Comparative Example 4 | 20 | −35 |
| Comparative Example 5 | 37 | −18 |

As can be seen from the results of Table 2, the cream of Example 1 using a combination of potassium cetyl phosphate as an anionic surfactant and crylate/C10-30 alkyl acrylate crosspolymer as a polymer having a lipophilic alkyl side chain showed a higher cream hardness by at least about 15 to 30 dyne/cm² than the creams of Comparative Examples 1 to 5 using a different surfactant and a different polymer.

### Experimental Example 2

### Evaluation of Cream Stability

The O/W type cosmetic creams prepared in Example 1 and Comparative Examples 1 to 5 were kept at the room temperature, at 45° C., or in a cycling constant-temperature chamber for 5 days, 10 days, 15 days, or one month to observe the cream stability based on hardness drop, suspension or separation of oil, etc. The results are presented in Table 3.

TABLE 3
Cream Stability over Temperature and Time

| | | | Example | Comparative Example | | | | |
|---|---|---|---|---|---|---|---|---|
| | | | 1 | 1 | 2 | 3 | 4 | 5 |
| Room | 5 | days | Good | Good | Good | Good | Good | Good |
| temp. | 10 | days | Good | Good | Good | Good | Good | Good |
| | 15 | days | Good | Good | Good | Good | H | Good |
| | 1 | month | Good | H | O** | Good | O* | H |
| 45° C. | 5 | days | Good | Good | Good | Good | Good | Good |
| | 10 | days | Good | Good | Good | Good | H | Good |
| | 15 | days | Good | Good | O* | Good | O* | Good |
| | 1 | month | Good | O* | O* | O* | O* | O* |
| Cycling | 5 | days | Good | Good | Good | Good | Good | Good |
| | 10 | days | Good | Good | Good | Good | O* | Good |
| | 15 | days | Good | Good | O* | Good | O* | Good |
| | 1 | month | Good | O* | O* | O* | O* | O* |

Note)
H: hardness drop
O*: oil suspension
O**: oil separation

As can be seen from Table 3, the O/W type cosmetic cream of Example 1 according to the present invention was superior in formulation stability to the O/W type cosmetic creams of Comparative Examples 1 to 5 at the room temperature, at 45° C. and in the cycling condition. In other words, the use of a combination of a polymer having a lipophilic alkyl side chain and an anionic surfactant in preparation of O/W type cosmetic creams contributed to enhanced cream hardness and higher formulation stability of the creams.

1. An O/W type cosmetic composition comprising, as an active ingredient for improving formulation stability:
a branched polymer having a lipophilic alkyl side chain; and
an anionic surfactanct.

2. The O/W type cosmetic composition as claimed in claim 1, wherein the branched polymer having a lipophilic alkyl side chain is selected from the group consisting of an acrylate/C10-30 alkyl acrylate crosspolymer, an acrylate/C12-22 alkyl methacrylate copolymer, an acrylate/beheneth-25 methacrylate copolymer, or an acrylate/ceteth-20 methacrylate copolymer.

3. The O/W type cosmetic composition as claimed in claim 1, wherein the anionic surfactant is selected from the group consisting of potassium cetyl phosphate, aluminum isostearyl glyceryl phosphate, or sodium lauryl sulfate.

4. The O/W type cosmetic composition as claimed in claim 1, wherein the branched polymer having a lipophilic alkyl side chain is contained in an amount of 0.05 to 0.5 wt. % with respect to the total weight of the composition.

5. The O/W type cosmetic composition as claimed in claim 1, wherein the anionic surfactant is contained in an amount of 0.5 to 2 wt. % with respect to the total weight of the composition.
